(19) United States
(12) Patent Application Publication
(10) Pub. No.: US 2023/0165702 A1
(43) Pub. Date: Jun. 1, 2023

(54) COUPLING SYSTEM

(71) Applicant: Ostique Limited, London (GB)

(72) Inventors: Stephanie MONTY, London (GB); Toni SCHNEIDER, London (GB); Austen Charles MILLER, Whitchurch, Hampshire (GB); Daniel PHILLIPS, Whitchurch, Hampshire (GB)

(73) Assignee: Ostique Limited, London (GB)

(21) Appl. No.: 17/922,396

(22) PCT Filed: Apr. 30, 2021
(86) PCT No.: PCT/GB2021/051057

§ 371 Date: Oct. 31, 2022

(30) Foreign Application Priority Data
Apr. 30, 2020 (GB) 2006409.3

(51) Int. Cl. A61F 5/448; A61F 5/44

(52) U.S. Cl. CPC A61F 5/448; A61F 5/4404; A61F 2005/4486

(57) ABSTRACT

A coupling system for use in medical applications is provided, comprising: a first member (5100) comprising a circular portion; and a second member (5240) being biased radially relative to the circular portion thereby to couple the first member (5100) to a further component (5200).

The present invention relates to a coupling system for use in medical applications. In particular, the invention relates to a coupling mechanism for use with an ostomy bag system.

Ostomy is a common surgical procedure during which an artificial opening, known as a stoma, is created for the discharge of bodily wastes. Patients who have undergone such a procedure, known as ostomates, typically wear products such as ostomy bags to collect the bodily wastes. The ostomate must empty and change the ostomy bag regularly, so it is important that this process is as comfortable and efficient as possible.

Aspects of the disclosure are set out in the accompanying claims.

In an aspect of the present disclosure, there is provided a coupling system for use in medical applications, comprising: a first member comprising a circular portion; and a second member being biased radially relative to the circular portion thereby to couple the first member to a further component (and/or to effect a coupling between the first member and the intermediate component).

In an aspect of the present disclosure, there is provided a coupling system for use in medical applications, comprising: a first member comprising a circular portion; and a further component comprising at least one second member being biased radially relative to the circular portion thereby to couple the first member to the further component.

This may provide a coupling system which is easier to attach, detach, and re-position, as the second member may be readily adjustable by a user by resisting the radial bias. As will be appreciated, since the second member is biased radially, the coupling may be adjusted from the side of the coupling system, which may provide for improved adjustment of the coupling when in situ. Re-positioning of the coupling may allow an associated ostomy bag to be located at different angles, which may improve comfort when e.g. sitting or driving.

The members are preferably separate, discrete and separable, but in an alternative may be connected. The circular portion is preferably circular in at least one dimension—i.e. it may be circular, tubular, and/or cylindrical. The first member and second member together form a part of the coupling, where the further component forms the other part. The second member is preferably configured to engage with the first member and further component to effect the coupling. The circular portion acts as a conduit in use—in other words, the coupling is between the circular portion and a further circular portion (of the further component). The coupling preferably allows the transfer of fluid therethrough.

Preferably, the second member is arranged to bear against at least one of the first member and a further component thereby to couple the first member to a further component; more preferably wherein the second member is arranged to bear against the first member and a further component thereby to couple the first member to a further component. The second member thereby acts as an intermediate component in the coupling. This may provide for improved adjustability and ease of use of the coupling as compared to a two-part coupling. By using a biased member that bears against two components, a simple, low-profile, and easily adjustable connection between the two components (i.e. the first member and the further component) may be provided. The second member preferably clips together with the first member. The first member preferably holds the first member in the correct position for engagement with a further component.

Preferably, the second member is biased radially outwards relative to the circular portion. This may allow the coupling to be easily adjusted or released in situ by depressing the second member radially inwardly.

Preferably, the second member acts as a spring. This may minimise componentry by eliminating the need for a separate biasing component. Preferably, the second member is shaped as a circlip. The circlip/second member acts as a spring, preferably wherein the circlip/second members does not require any additional elastic components (e.g. additional springs or fasteners).

Preferably, the second member extends at least partially around the first member. This may provide a low-profile coupling, since the second member can encircle the circular portion of the first member.

In a further aspect of the present disclosure, there is provided a spring shaped as a circlip for a coupling component for use in medical applications. The spring is preferably biased outward relative to the arc of the circlip.

Preferably, the second member/spring further comprises handles which protrude from the coupling system/spring; preferably wherein the handles are located proximate respective ends of the second member/spring, more preferably such that the handles can be pinched to compress the second member/spring. This may improve the ease of adjusting or releasing the components.

Preferably, the handles comprise cut-out portions such that other components of the coupling system extend through the cut-out portions. This may assist in keeping the size of the coupling small, while still allowing the handles to protrude.

Preferably, the second member/spring comprises first and second formations for engaging with different components thereby to couple the first member/a component to a further component, preferably wherein the first and second formations are provided on a side of the second member/spring, more preferably wherein the first and second formations are adjacent. This may provide for compact engagement means. The first and second formations preferably extend around the second member/spring. The formations bear against the components as a result of the biasing of the second member. The components that the formations engage with are the first member and the further component.

Preferably, the first and second formations are arranged at different widths on the second member/spring. In other words, the first and second formations have different diameters (relative to the arc of the second member/spring and/or the circular portion). This may allow for convenient engagement with different components.

Preferably, at least one of the first and second formations comprise teeth, the teeth forming part of a ratchet mechanism. Preferably only one of the first and second formations comprise teeth, more preferably the formation that engages with the further component. This may allow for rotation between the second member/spring and further component when disengaged (i.e. when the second member is compressed), without allowing rotation when engaged. The other formation is preferably a ridge on the second member—the teeth are preferably mounted on a further ridge.

Preferably, the first member comprises at least one formation with which the second member engages, the at least one formation being located outwardly of the circular portion. This may allow for engagement without interfering with the circular portion.

Preferably, the at least one formation comprises at least one clip, preferably wherein the at least one clip extends outwardly from the circular portion; more preferably wherein the width of the at least one clip is configured to allow compression of the second member. The first member preferably further comprises a further flange which acts as a base portion.

Preferably, the first and second members comprise further cooperative formations which engage thereby to prevent relative rotation between the first and second members.

Preferably, the coupling system further comprises a third member comprising a circular portion, optionally wherein the third member is the further component; preferably wherein the circular portion is arranged to form a continuous conduit with the circular portion of the first member.

Preferably, the third member further comprises at least one formation against which the second member bears. Preferably, the formation comprises (or is included on) an outer wall, more preferably wherein the outer wall is located outwardly of the conduit. Preferably, the outer wall comprises teeth forming part of a ratchet mechanism (more preferably wherein said teeth engage with corresponding teeth on the second member).

Preferably, the third member further comprises a base, more preferably extending between the circular conduit and at least one formation, for connecting to a user's skin, more preferably via adhesive.

Preferably, a width of the base is configured such that, when compressed, the first and second member can be rotated within a channel created by the gap between the conduit and the at least one formation.

Preferably, the coupling system is for an ostomy bag, wherein the first member attaches to an ostomy bag. Preferably, the third member/further component attaches to a user's skin.

In a further aspect of the present disclosure, there is provided an ostomy bag system, comprising the coupling system as described herein, and an ostomy bag. The ostomy bag system preferably further comprises a cover for the bag; more preferably wherein wherein the cover is configured to be suspended from the coupling system of the bag; more preferably wherein the cover comprises formations (such as pockets) for receiving corresponding formations of the coupling system; yet more preferably wherein said corresponding formations are handles of the coupling system (optionally the handles of the second member as described herein). Alternatively, the cover may surround the bag and may comprise an opening for insertion of the bag; wherein the cover may comprise pockets for receiving handles of the coupling system.

In a further aspect of the present disclosure, there is provided a cover for surrounding an ostomy bag, comprising an opening through which the bag can be inserted. The cover may include pockets for receiving handles of a coupling system associated with the bag.

In a further aspect of the present disclosure, there is provided a coupling system for use in medical applications, comprising: a first member; and a second member being biased relative to the first member thereby to couple the first member to a further component.

In a further aspect of the present disclosure, there is provided a coupling system for use in medical applications, comprising: first and second coupling members; and an intermediate member which couples the first and second coupling members.

In a further aspect of the present disclosure, there is provided an ostomy coupling being releasable from the side of the coupling. Preferably, the coupling is releasable upon application of pressure in a direction other than towards the human body. Preferably, the coupling is releasable via a coupling component including handles, more preferably wherein such handles may be pinched to release the coupling.

In an alternative, the first and second member may be connected by a plurality of flexible ties, preferably wherein the second member substantially encircles the first member.

In an alternative, formations on the second member may engage with corresponding formations on a further component thereby to couple the first member to a further component, preferably wherein the second member is arranged to clip the first member to the further component; more preferably wherein the second member pivots relative to the second member thereby to engage with the further component.

In an alternative, formations on the second member may engage with corresponding formations on a further component thereby to couple the first member to a further component, preferably wherein the second member engages with the further component via a screw fitting thereby to couple the first member to a further component.

In an alternative, formations on the second member may engage with corresponding formations on a further component thereby to couple the first member to a further component, preferably wherein the second member folds around the first member thereby to clip the first member to the further component.

Preferably, the system further comprises the further component, wherein the further component comprises the second member. By providing a biased member on the further component to which the first member is connected, a simpler and more robust system may be provided.

Preferably, the further component further comprises a circular portion; and the second member is an arm extending from the circular portion. Use of a biased arm may provide a relatively strong biasing force in a small area, and may improve ease of use of the system (since the arm may be deflected).

As will be appreciated, the second member is also biased relative to the circular portion of the further component.

Preferably, the arm is arranged radially outward of the circular portion. This may provide for a larger fluid conduit.

Preferably, at least part of the arm extends generally tangentially to the circular portion. More preferably said extension is in a direction the same as a direction in which the arm is rotated in order to couple the first member to a further component. This may assist in reducing the form factor of the system.

Preferably, the coupling system further comprises a ring component, wherein the second member is configured to bear against the ring component to couple the first member and the further component. Use of such an intermediary component may provide for a robust connection between the first member and further component.

Preferably, the ring component is arranged to rotate relative to the further component. This may improve ease of coupling and decoupling.

Preferably, the ring component comprises a formation for receiving the second member thereby to couple the first member and the further component. More preferably the ring component is arranged to be rotated relative to the further component into a position in which the formation receives the second member. This may improve ease of coupling and decoupling.

Preferably, the formation receives the second member in a snap fit. More preferably wherein the formation and second formation are configured to produce an audible sound when the formation receives the second member. Even more preferably wherein the system produces a visual indication when the formation receives the second member. This may assist a user in using the system.

Preferably, the ring component comprises a flange including the formation, the flange being configured to accommodate and deflect the second member. This may improve ease of use, since a user simply needs to rotate the ring component to deflect the braised arm via the flange. More preferably the formation is a groove or discontinuity in the flange. Even more preferably a groove or discontinuity in an outer wall of the flange.

Preferably, the further component further comprises a tab located proximate the second member, wherein the flange comprises a further tab configured to abut the tab thereby to prevent rotation of the ring component relative to the further component beyond a position at which the formation receives the second member. More preferably the tab is located adjacent an end of the second member. Even more preferably wherein the tab is located in a direction relative to the second member that is the same as a direction in which the arm is rotated in order to couple the first member to a further component. This may improve useability.

Preferably, the second member comprises a protrusion extending radially from the second member, wherein said protrusion is arranged to be received by the formation. More preferably said protrusion is proximate an end of the second member. This may provide for a robust connection,

Preferably, the ring component is retained by the first member. More preferably such that the ring component cannot move in an axial direction relative to the first member.

Preferably, the ring component is retained such that the ring component can rotate with respect to the first member. More preferably the ring component is retained in a groove of the first member. This may improve ease of use of the system.

Preferably, wherein the ring component comprises at least one handle. More preferably the at least one handle and the further tab are continuous.

Preferably, the coupling system further comprises a plurality of second members. More preferably it further comprises a plurality of corresponding formations. Even more preferably it comprises a plurality of tabs. This may improve the robustness of the coupling.

Preferably, the coupling system further comprises three second members being arranged symmetrically about the circular portion.

The flange may be discontinuous, preferably such that the continuities correspond to the locations of the second members in a position in which the formations(s) receive the locking members. More preferably thereby to allow the further component to be removed from the first member.

Preferably, the first member and further component couple such that a fluid conduit is formed.

Preferably, one or both of the first member and further component comprises a deformable element being configured to be compressed upon coupling to form a seal.

Preferably, the first member and further component are prevented from rotating relative to each other when coupled, more preferably by friction in the seal.

Optionally, the first member and further component are able to rotate relative to each other.

Optionally, the first member and further component are operable to be coupled in a first mode in which the first member and further component are prevented from rotating relative to each other; and a second mode in which the first member and further component are able to rotate relative to each other. More preferably: wherein a variant second member and/or further component is provided for use in the second mode. Even more preferably wherein said variant second member and/or further component has one or more different material properties to the second member and/or further component, and/or wherein the second member is actuable thereby to allow control of the tightness of the seal. Such modes may be respectively used e.g. during the day (when it may be convenient that the system—and thereby an ostomy bag—is fixed in position, and at night, where for comfort it may be desirable that the system—and thereby an ostomy bag—is free to move while remain attached.

Preferably, the second member is biased radially outwards relative to the circular portion.

As used herein, the term “spring” preferably connotes an elastic object that stores mechanical energy.

As used herein, the term “circlip” preferably connotes a semi-flexible ring with open ends, preferably having an arcuate shape.

The invention extends to methods and/or apparatus substantially as herein described with reference to the accompanying drawings.

The term ‘comprising’ as used in this specification and claims preferably means ‘consisting at least in part of’. When interpreting statements in this specification and claims which include the term ‘comprising’, other features besides the features prefaced by this term in each statement can also be present. Related terms such as ‘comprise’ and ‘comprised’ are to be interpreted in a similar manner.

Any apparatus feature as described herein may also be provided as a method feature, and vice versa.

Any feature in one aspect of the invention may be applied to other aspects of the invention, in any appropriate combination. In particular, method aspects may be applied to apparatus aspects, and vice versa. Furthermore, any, some and/or all features in one aspect can be applied to any, some and/or all features in any other aspect, in any appropriate combination.

It should also be appreciated that particular combinations of the various features described and defined in any aspects of the invention can be implemented and/or supplied and/or used independently.

These and other aspects of the present invention will become apparent from the following exemplary embodiments that are described with reference to the following figures in which:

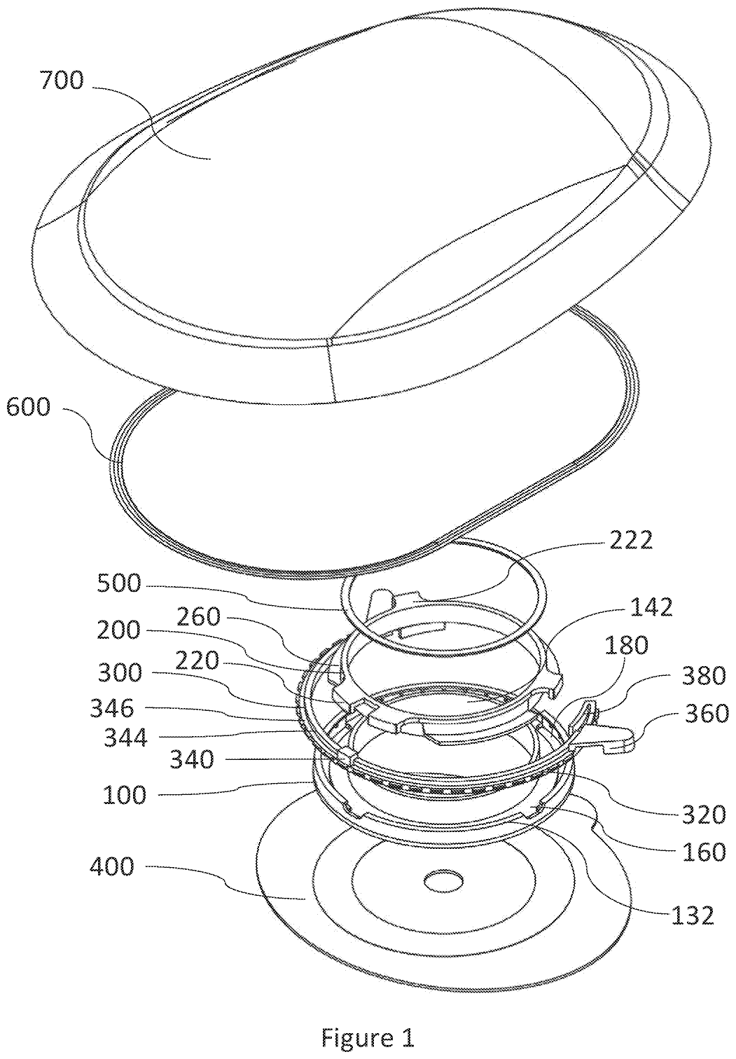

FIG. 1 shows an exploded view of an example of the present invention;

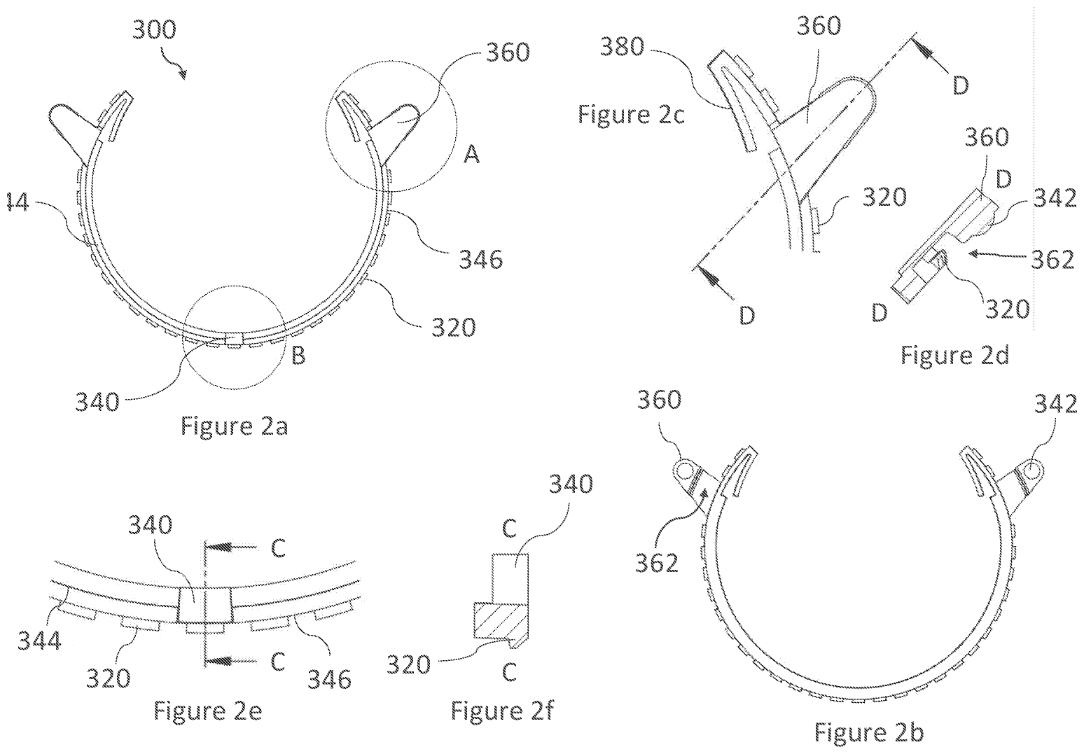

FIGS. 2a to f show schematic top, rear and detail views of a spring clip;

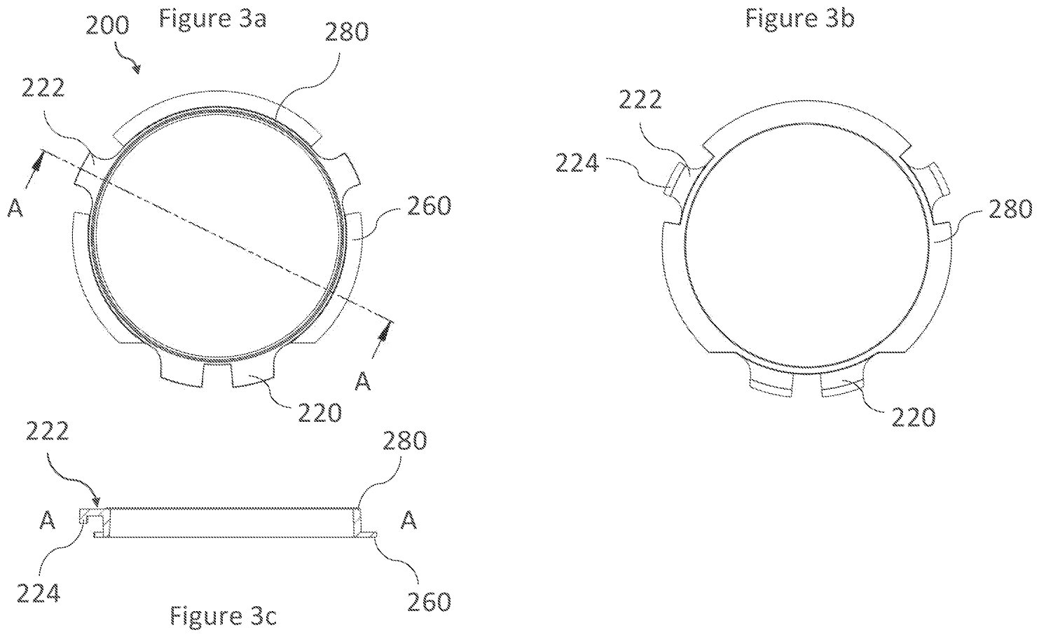

FIGS. 3a to c show schematic top, rear and cross-sectional views of a latch retainer;

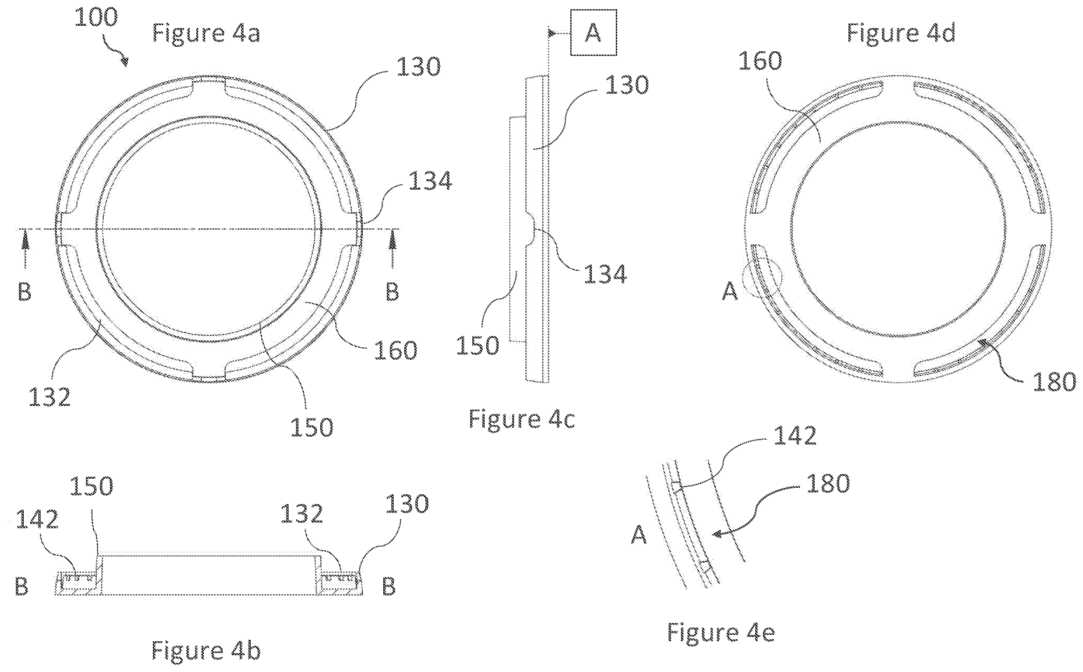

FIGS. 4a to e show schematic top, cross-sectional and detail views of a baseplate;

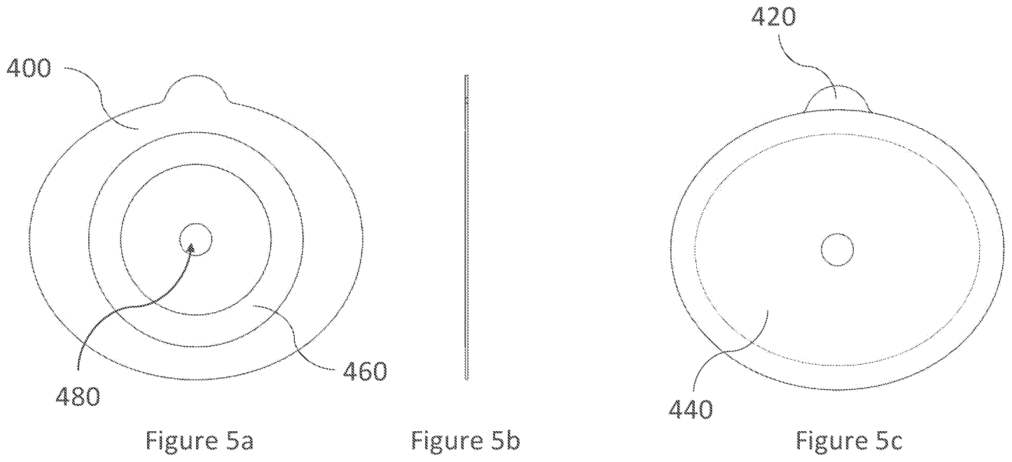

FIGS. 5a and b show schematic top and rear views of a wafer;

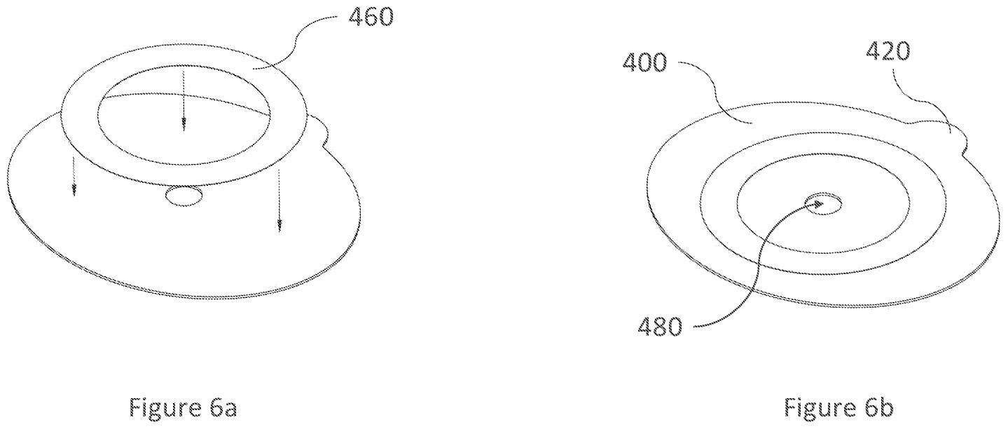

FIGS. 6a and b show perspective schematic views of the wafer;

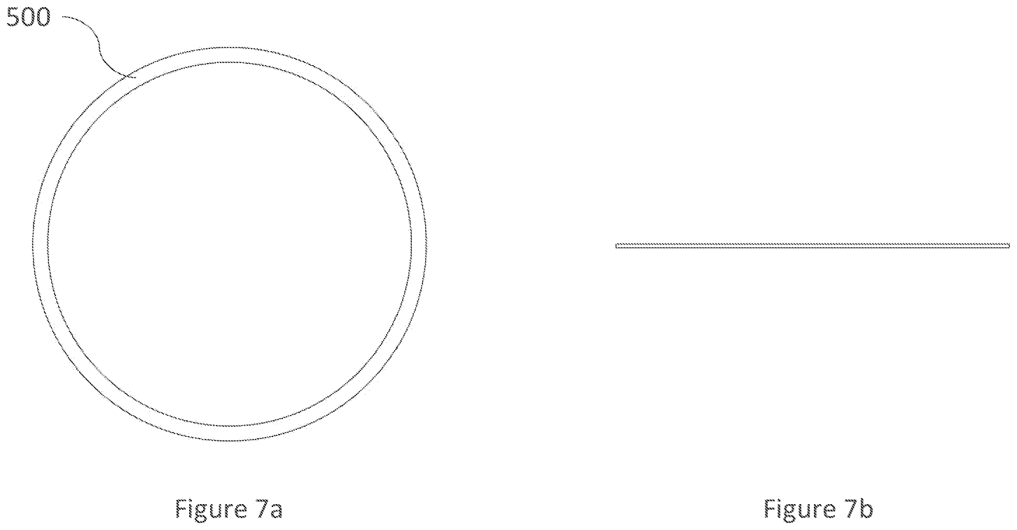

FIG. 7 shows schematic top and side views of a collection bag adhesive;

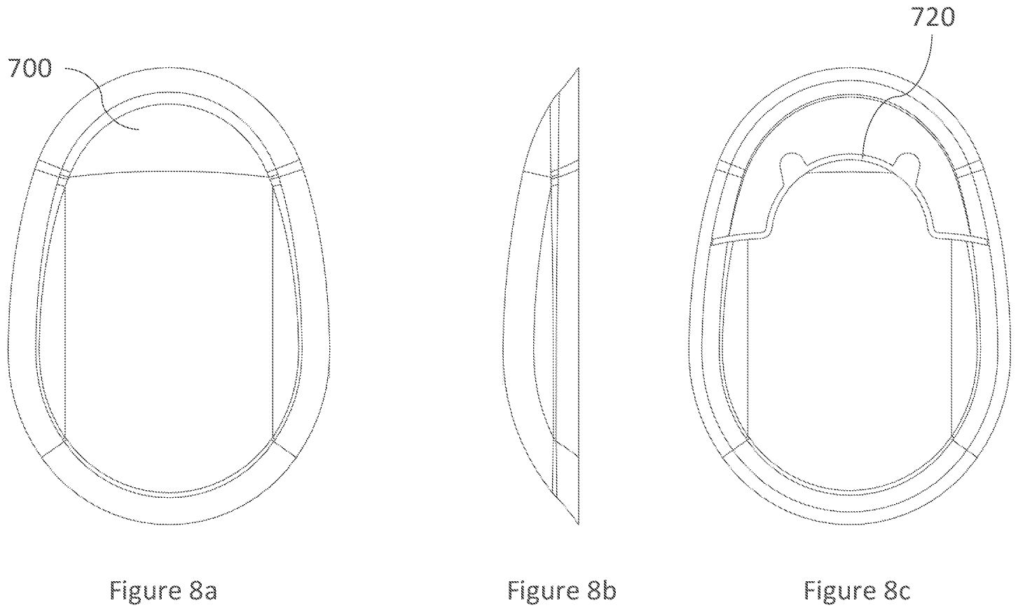

FIGS. 8a to c show schematic top, side and rear views of an example of a cover;

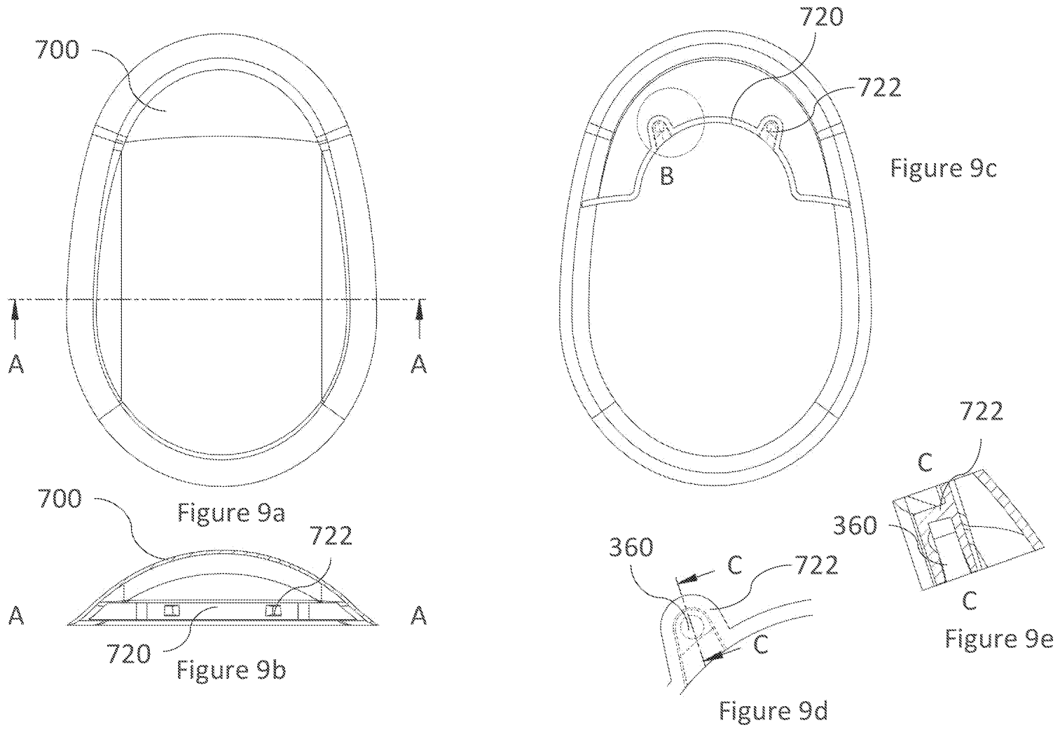

FIGS. 9a to e show schematic views of a cover with reference to a coupling system;

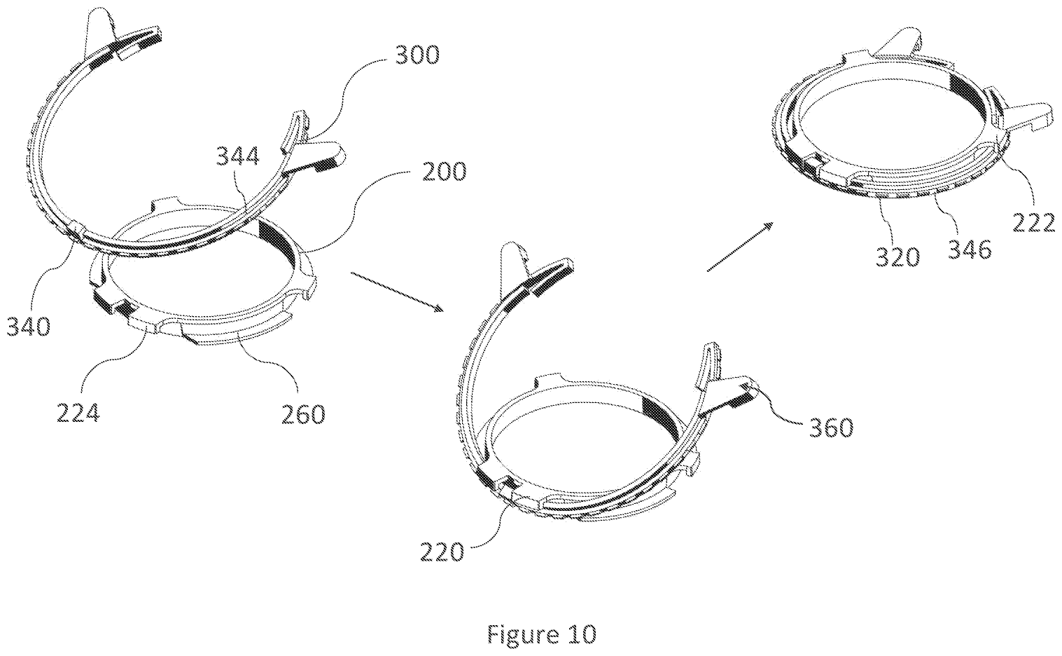

FIG. 10 shows schematic views of the steps of attachment of the spring clip and latch retainer of an example of the invention;

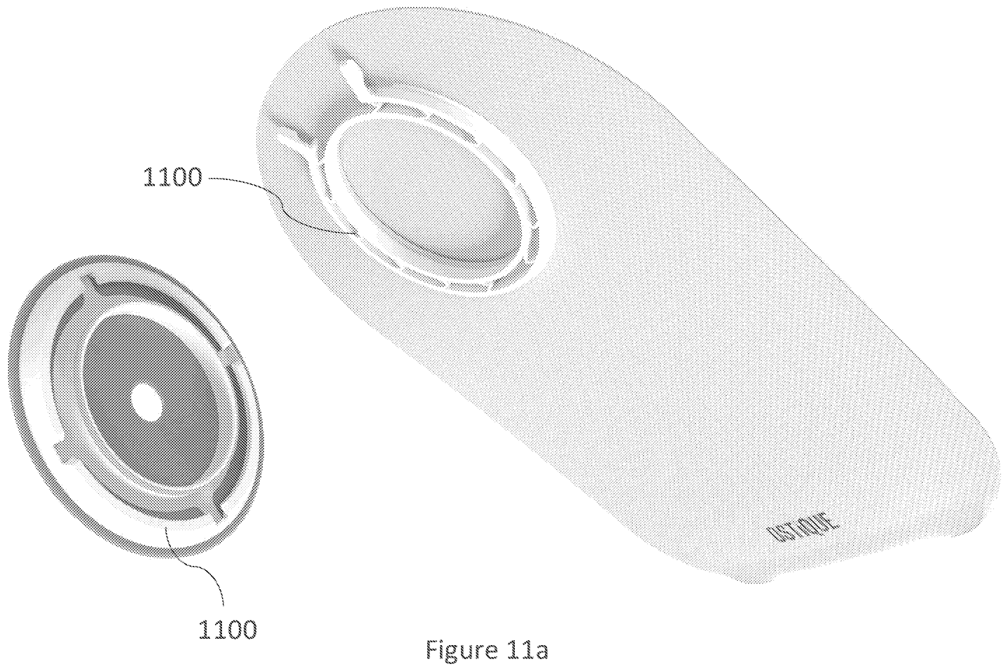

FIGS. 11a to e show schematic views of an alternative example of the present invention;

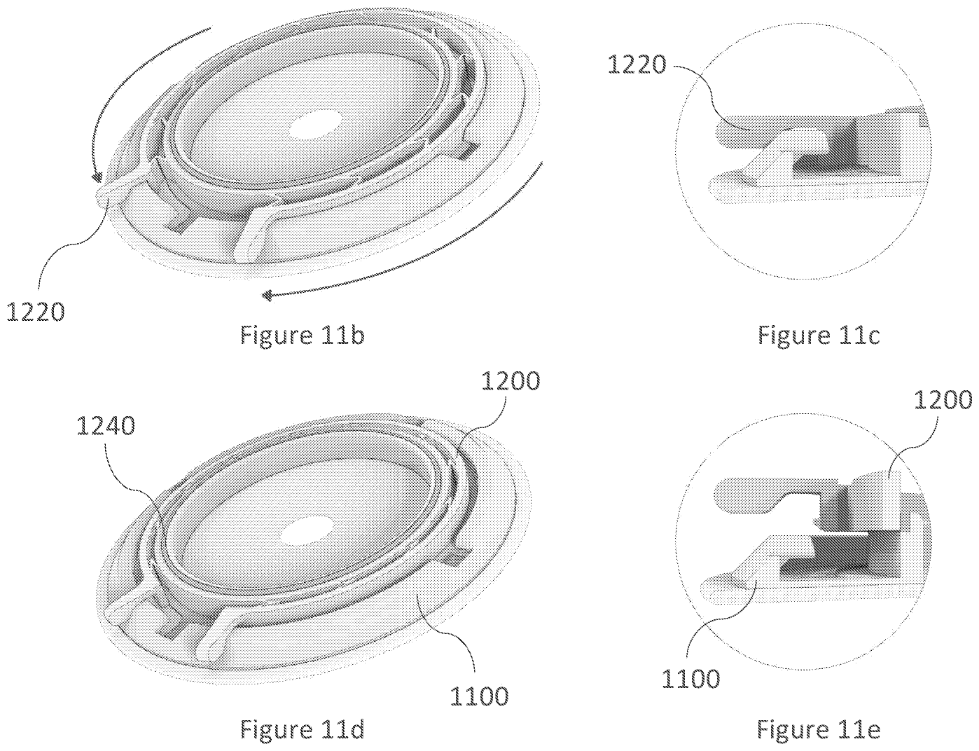

FIGS. 12a to c show schematic views of a further alternative example of the present invention;

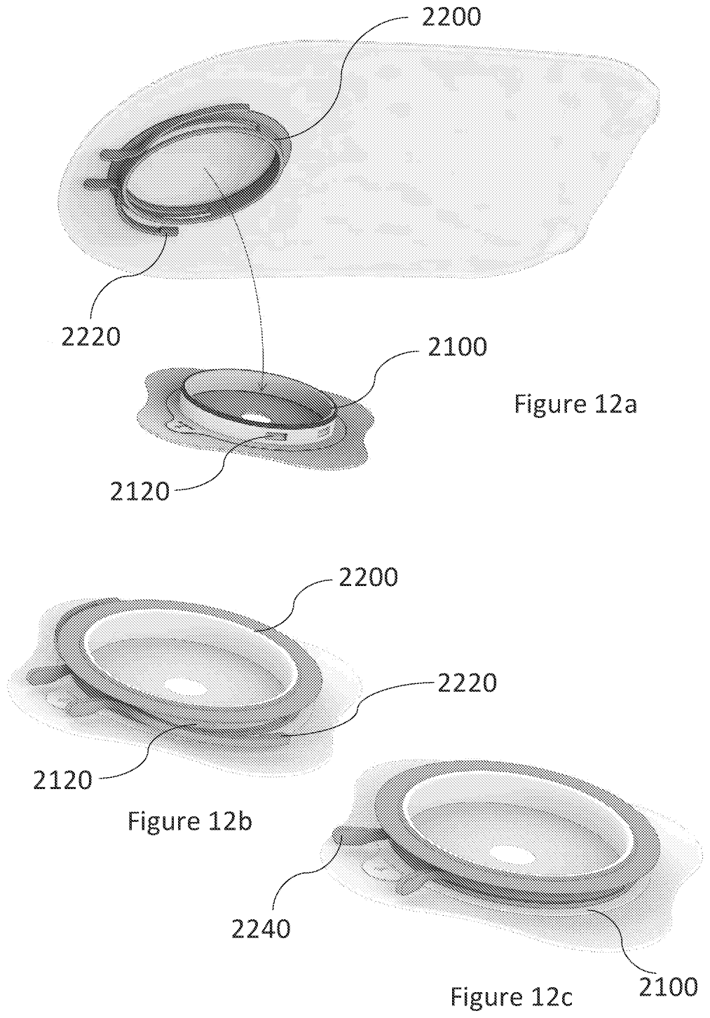

FIGS. 13a and b show schematic views of a further alternative example of the present invention;

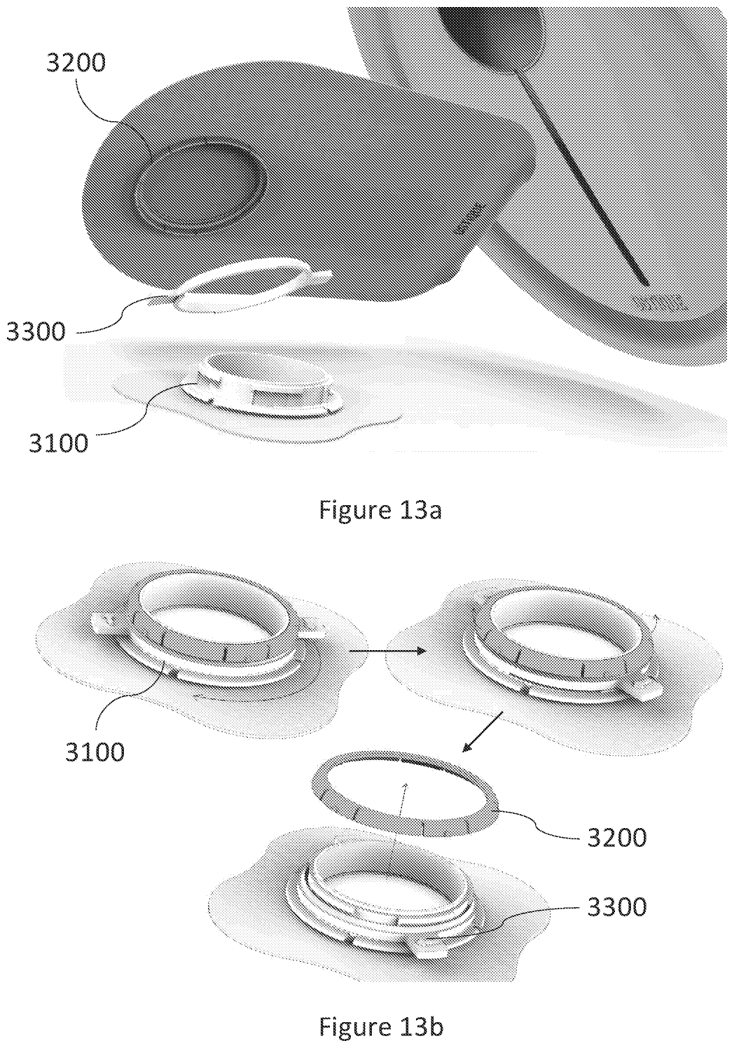

FIGS. 14a to c show schematic views of a further alternative example of the present invention;

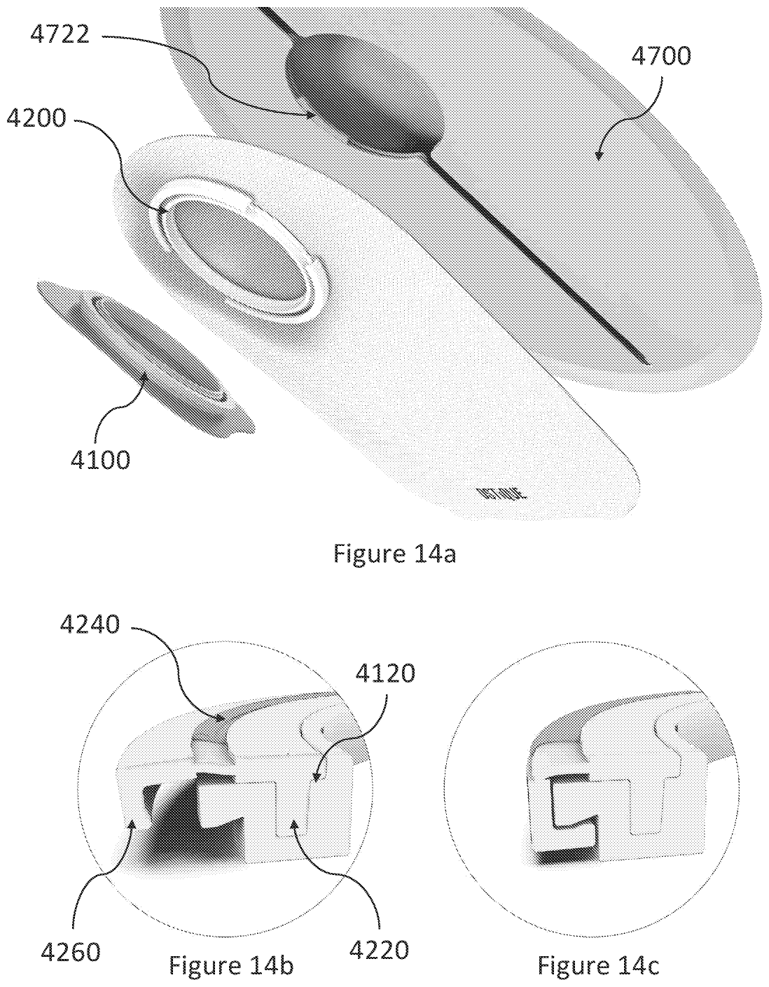

FIG. 15 shows various further alternative mechanisms;

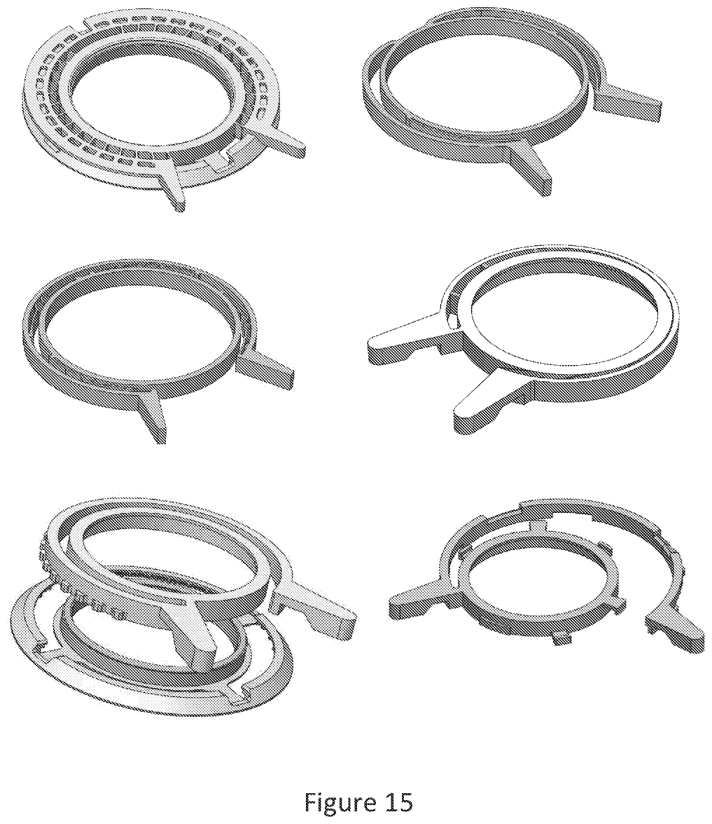

FIG. 16 shows an exploded view of a further example coupling system of the present invention;

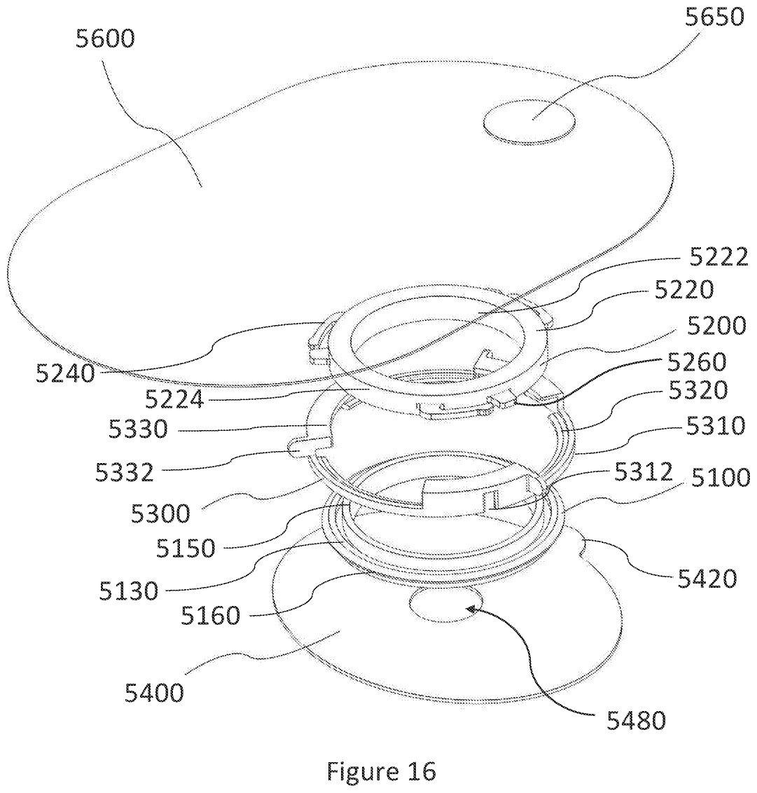

FIGS. 17a to c show schematic perspective views of wafer, baseplate and rotating ring components;

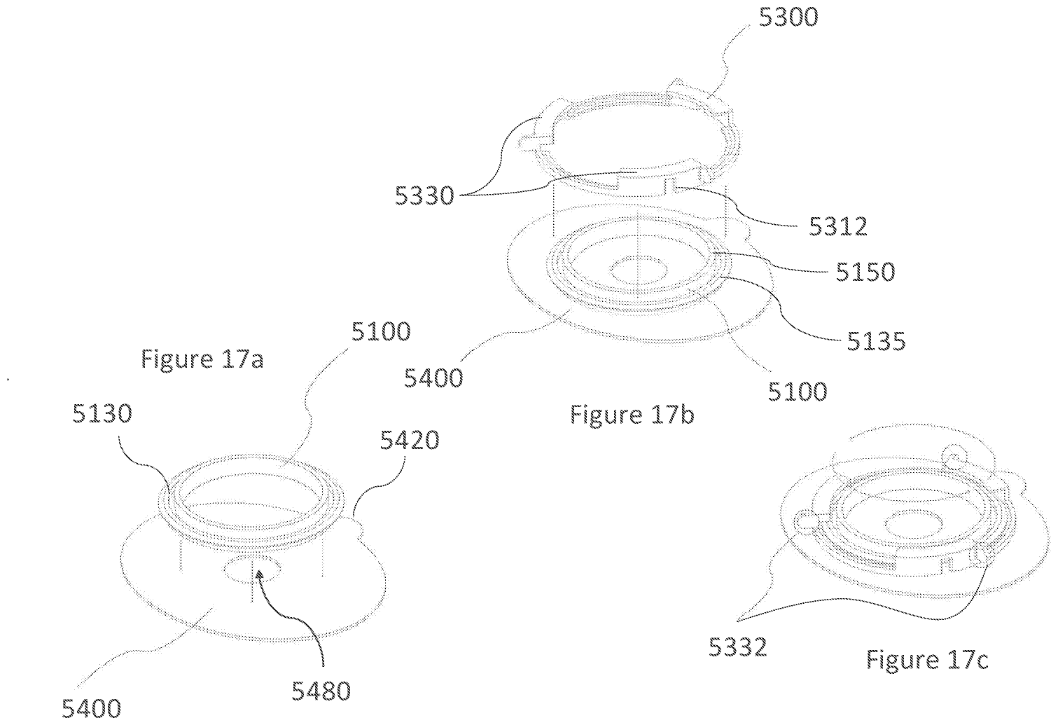

FIGS. 18a and b show schematic perspective views of bag and bag clip components;

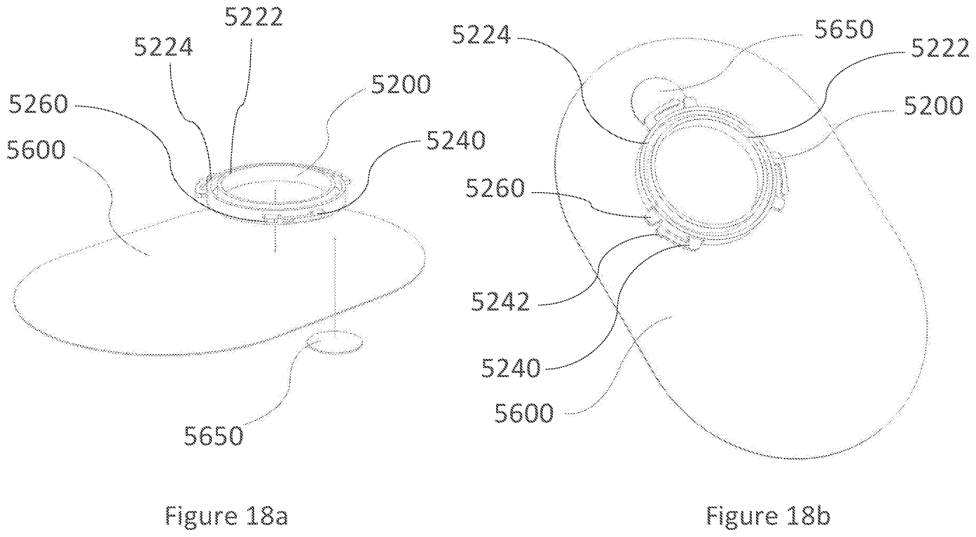

FIG. 19 shows a schematic perspective view of the attachment of the wafer, baseplate and rotating ring and bag and bag clip;

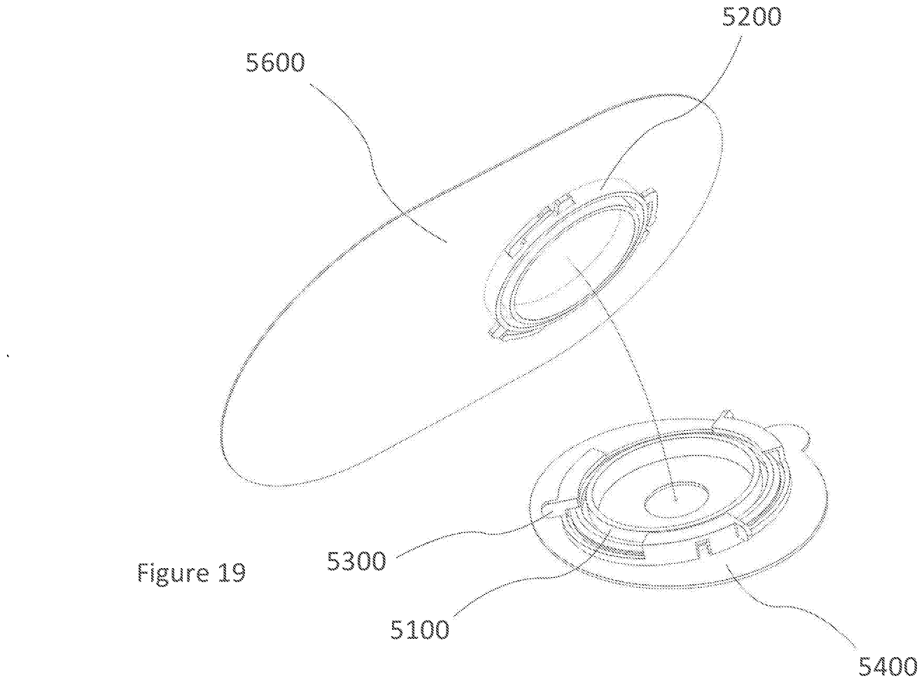

FIGS. 20a and b show schematic top views of the connection of the bag clip and rotating ring;

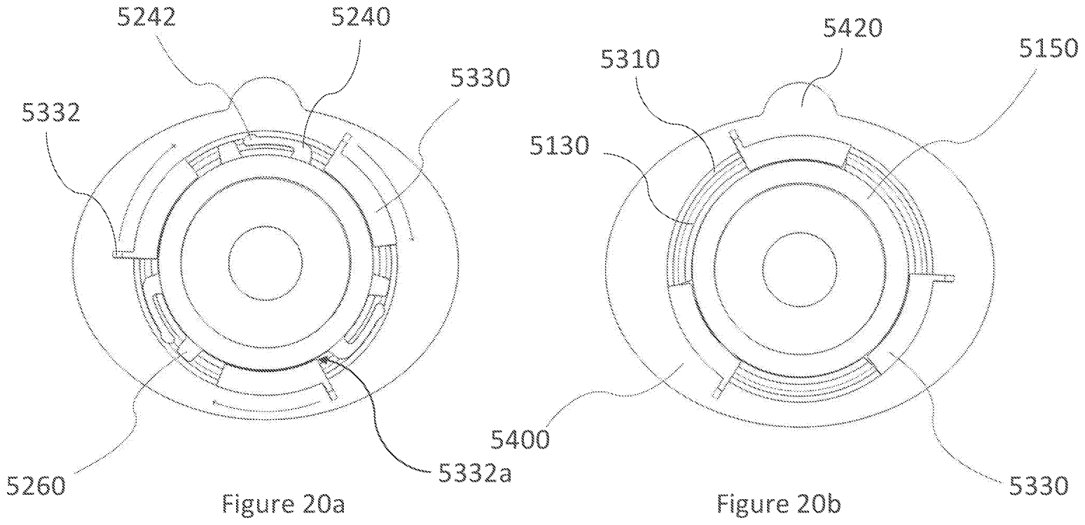

FIGS. 21a and b show schematic side and bottom detail views of the connection of the bag clip and rotating ring;

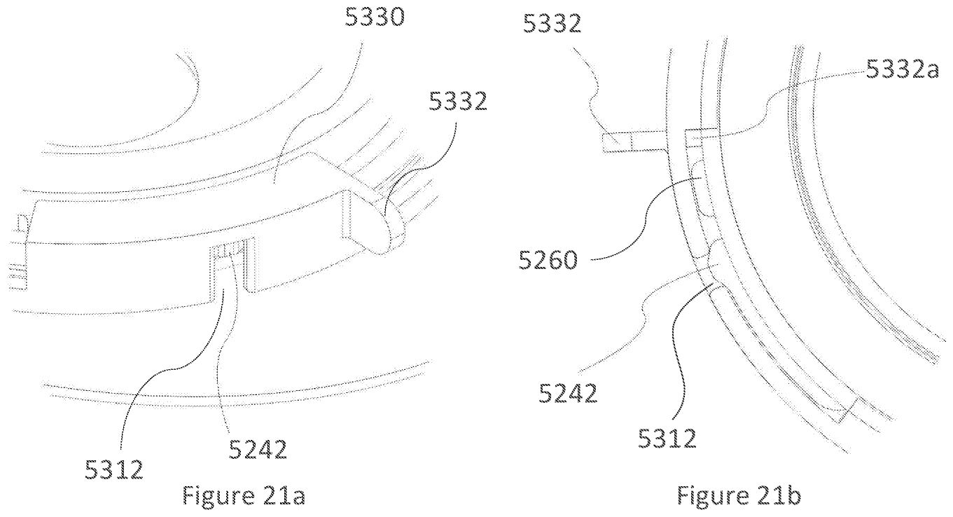

FIGS. 22a to c show schematic side, cross section and detail views of the connection of the bag plate, rotating ring and baseplate;

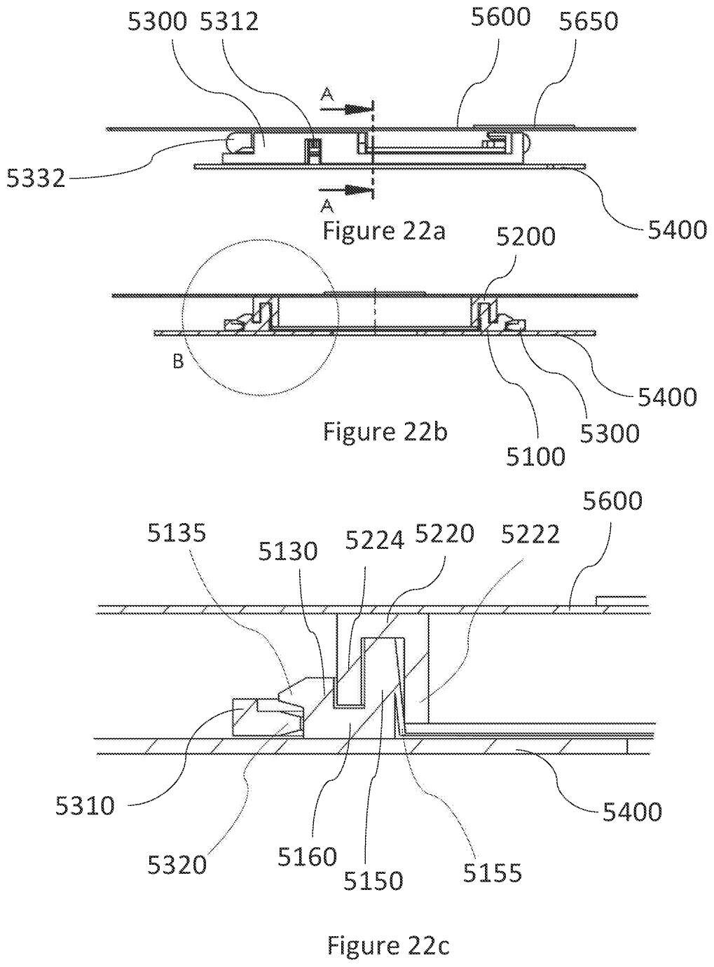

FIG. 23 shows a schematic perspective view of a cover with reference to the coupling system;

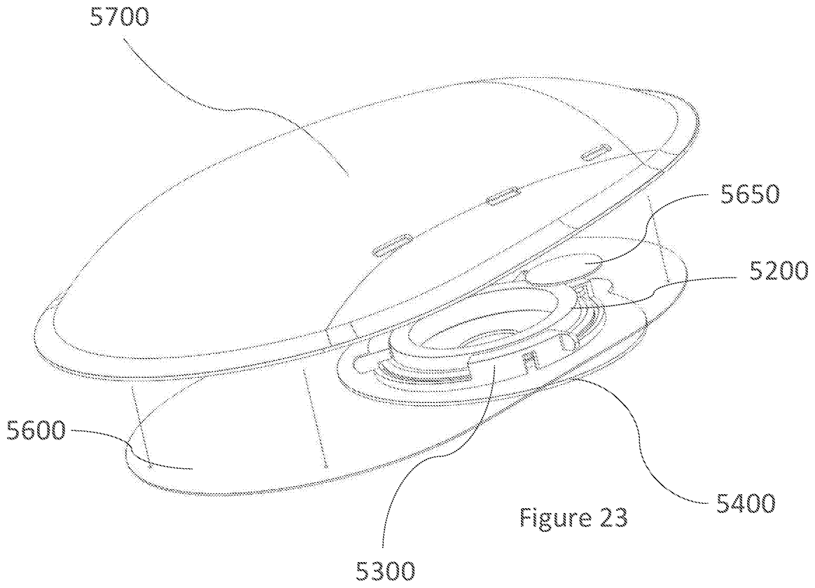

FIG. 24 shows an exploded view of a yet further example coupling system of the present invention; and

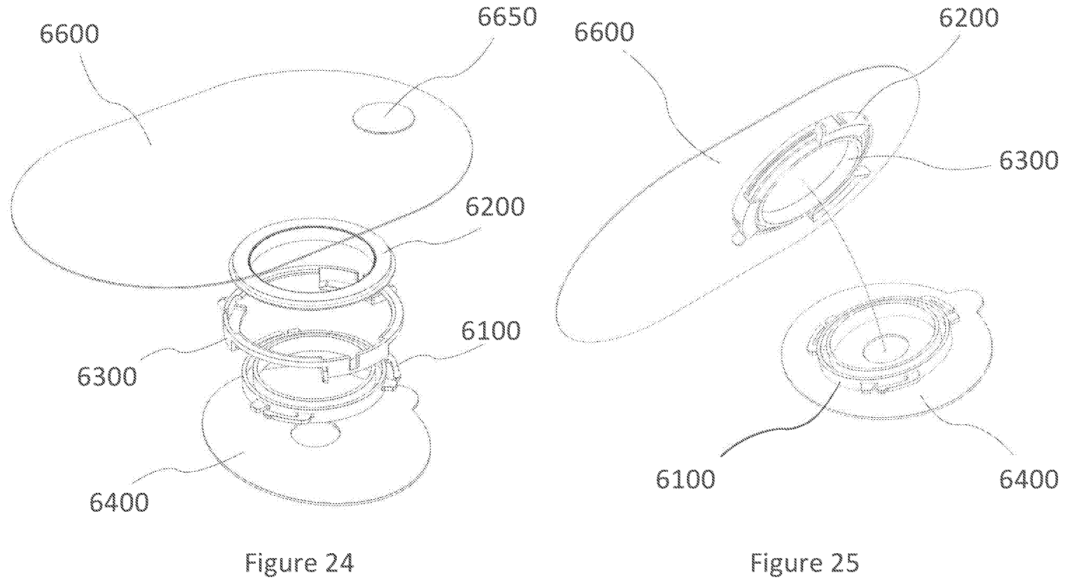

FIG. 25 shows a schematic perspective view of the connection of the system of FIG. 24.

### SPECIFIC DESCRIPTION

FIG. 1 provides an exploded view of an example of an ostomy bag system including the coupling system of the invention. The ostomy bag system comprises a base plate 100, a latch retainer 200, a spring clip 300, wafer 400, (optionally) collection bag adhesive 500, collection bag 600, and a cover 700. Both the base plate 100 and the latch retainer 200 are formed as circular components (i.e. they include circular portions), which connect together to form a conduit. In other words, a coupling is provided between the base plate 100 and the latch retainer 200. The coupling between the base plate 100 and the latch retainer 200 is provided by the spring clip 300, which is formed as a circlip and exerts pressure against both the base plate 100 and the latch retainer 200 (i.e. radially away from the circular parts of base plate 100 and the latch retainer 200). The base plate 100 is attachable to the human body over a stoma. The collection bag is attached to the latch retainer 200 by, for example, adhesive 500. As will be appreciated, the present invention provides a releasable coupling via the spring clip 300.

The latch retainer 200, the spring clip 300 and the base plate 100 may alternatively be referred to, respectively, as the first, second and third members.

FIGS. 2a and b provide top and bottom views of the spring clip 300. The spring clip 300 of the present invention is a long, thin elastic component which is curved into an arcuate shape. The spring clip 300 is configured to at least partially extend around (a circular portion of) the latch retainer 200. Generally, the spring clip 300 extends around approximately ⅔ of the circular portion of the latch retainer. The spring clip 300 is formed as a circlip, which is urged outwardly (i.e. away from the circular portion of the latch retainer 200, when in use).

As can be most easily seen in FIG. 1, the spring clip 300 comprises an outer surface 344 which faces outwardly (away from the interior of the spring clip). The outer surface 344 comprises a shoulder (or flange) 346, which takes up a lower half of the outer surface 344 and extends a short way away from the outer surface 344. This provides the spring clip 300 with two outwardly facing surfaces (i.e. the shoulder 346 and the top part of the outer surface 344), which act as engagement formations in use—that is, the surfaces bear against components in order to effect the engagement.

The spring clip 300 comprises teeth 320 spaced uniformly around the shoulder 346. The teeth 320 may be spaced at any angle which is a factor of 360 degrees. Additionally, there is provided a further protrusion 340 on the spring clip 300. The further protrusion 340 is a cuboid feature which extends from the shoulder 346 and the top surface of the spring clip 300. The further protrusion 340 is located generally in the middle of the spring clip 300, opposite the opening in the spring clip 300 defined by its ends. In use, the further protrusion acts to locate the spring clip 300 relative to the latch retainer 200.

FIG. 2e shows a magnified view of the area marked B in FIG. 2a, illustrating the protrusion 340 at the edge of the spring clip 300. FIG. 2f provides a cross-sectional aspect along line C-C. As shown in FIG. 2f, the teeth 320 comprise a sloped lower edge in order to facilitate placement within the base plate 100.

Handles 360 are provided proximate respective ends of the spring clip 300. The handles 360 extend from the outer surface 344 in the plane of the circular arc of the spring clip 300. The handles 360 are angled slightly towards the ends of the spring clip 300. The handles 360 have a generally triangular shape, albeit rounded at their ends. The handles 360 may optionally comprise grip formations, for example domed protrusions and/or a rubber grip casing. The handles 360 comprise generally square cut-out portions located on their underside, configured to fit other components of the system such as the base plate 100. The provision of the cut-out portion also allows the row of teeth 320 to extend around the flange without interruption. One such handle can be seen in FIG. 2c, which provides a magnified view of the area marked A in FIG. 2a. FIG. 2d provides a cross section across the line D-D, which in particular shows the cut out portion and a tooth 320 extending from the flange into the cut out portion, generally towards the end of the handle 360. The underside of the handle 360 includes a raised portion 342 to assist in gripping the handle 360.

The spring clip 200 further comprises spacer springs 380, which are provided on the end of the spring clip 300 (and so proximate the handles 360). The spacer springs 380 extend from the end of the spring clip 300 back towards the main part of the spring clip, on the inside of the spring clip 300 (relative to the arc shape defined by the spring clip 300). The spacer springs preferably extend generally as far as the handles 360. The spring clips 380 have a radial bias contrary to that of the spring clip 300 as a whole—in the present example therefore they have an inward radial bias. A small cut out portion on the inside of the spring clip 300 is provided for fitment of the spacer spring 380 when compressed. The spacer springs 380 are configured to maintain contact with the latch retainer 200 when the spring clip 300 and latch retainer 200 are connected.

FIGS. 3a, b and c provide representations of an example of the latch retainer 200 from a top view, a cross sectional view along A-A, and a rear view. The latch retainer 200 comprises a central circular (or ring-shaped) portion 280, from which extends a flange 260. The flange 260 is located at the base of the central circular portion 280. The flange 260 is not continuous but instead includes feature cut-out portions. The edges of the cut-out portions may be at an angle rather than perpendicular to the radial orientation of the central circular portion 280.

Further formations are provided at the top of central circular portion 280, in the form of a plurality of clips 220, 222. Specifically, four clips are provided, where two rear clips 220 are located close together on the circular portion 280. Two front clips 222 are each provided generally one third of the way around the circular portion 280 from the two rear clips, such that the circular portion is roughly divided into thirds by the two rear clips 220, and each of the front clips 222. The clips 220, 222 each comprise a flange portion projecting from the circular portion 280, and a lip 224 which project in the same direction as the circular portion 280, thereby to form circular sections at a wider radius than the circular portion 280. The cut-out portions of the flange 260 are located to coincide with the clips 220, 222. FIG. 3c provides a cross-sectional view across the diameter of the latch retainer 200, showing the clip 222 at one end and the flange 260 at the opposite end.

FIGS. 4a, b, c, d, and e show an example of the base plate 100. The base plate 100 (also referred to as the third member) comprises a generally circular portion, which is configured to engage with the circular portion of the latch retainer to provide the coupling. The base plate 100 is configured to be attachable to the human body over a stoma (as one example of a potential use). The base plate 100 and the latch retainer 200 are in this case configured to form a conduit between the stoma and a collection bag, where the base plate 100 and the latch retainer 200 are connected by the spring clip 300.

FIG. 4a provides a top view of the base plate 100, which comprises an inner circular (or tubular) component 150, configured to form a continuous conduit with the latch retainer 200; a base 160, and an outer wall 130, which is located outwardly of inner circular component 150. The base 160 is a flat, generally ring-shape part which extends from the bottom of the inner circular component 150 to the outer wall 130, thereby to connect them. The outer wall 130 extends less far from the base 160 than the inner circular component 150. The outer wall 130 further comprises an upper lip 132, which extends radially inward in relation to the inner circular component 150 (part of way across the base 160). The upper lip 132 may be formed as a number of sections (specifically, four), between which are spacings 134. FIG. 4b provides a side view of the base plate across the line B-B, which intersects two such spacings 134 on either side of the base plate 100. The outer wall 130 has a lower height by the spacings 134—that is, the outer wall 130 includes dips which are aligned with the spacings 134. FIG. 4c provides a side view of the base plate 100.

The outer wall 150 further comprises uniformly spaced teeth 142 on the inside of the outer wall 150, which are configured to form a ratchet mechanism with the spring clip teeth 320. The teeth 142 may be spaced at any angle which is a factor of 360 degrees. The width of the base 160 is configured such that, when compressed, the spring clip 300 and latch retainer 200 may be rotated in the channel between the inner circular component 150 and the ratchet mechanism. This rotation is preferably possible in both directions.

The base 160 further comprises generally arcuate cut-out portions 180 on the underside of the base 160, which are aligned with the upper lip 132. FIG. 4d illustrates an example of such cut-outs 180, while FIG. 4e provides a detailed view of section A in FIG. 4d.

FIG. 5a provides a top view of the wafer 400, which is the side in contact with the baseplate 100. FIG. 5b provides a bottom view of the wafer 400, which is the side in contact with skin. The wafer 400 is a thin component formed of fabric or plastic, which is attached to the user's skin via adhesive on one side and the base plate 100 on the other side. A double-sided adhesive strip 460 is used to attach the base plate adhesive 400 to the base plate 100. Perspective views of this arrangement are provided in FIGS. 6a and b. Adhesive 440 is provided over a substantial portion of the bottom side of the wafer, covering all of the wafer 440 apart from the edges and a formation 420 at the outer edge of the wafer 400, which may be used during removal. As the system is configured to receive a stoma, there is provided a corresponding opening 480. The size and shape of this opening 480 may be determined and adapted according to the needs of the user.

FIGS. 7a and b provide top and side views of the optional collection bag adhesive 500. This is formed as a double-sided adhesive ring which connects the latch retainer 200 with the ostomy bag 600. In an alternative, the latch retainer 200 is heat welded to the ostomy bag 600.

The ostomy bag system also includes the ostomy bag 600 itself, which is configured to receive bodily wastes. The ostomy bag includes an opening for receiving waste from the stoma, which is engaged with the latch retainer 200 to receive waste therefrom. The ostomy bag 600 is preferably formed to have a shallow depth, such that it may sit discretely against the body.

The ostomy bag 600 may be covered during normal use, so that the system may be as discrete as possible. FIGS. 8 a, b and c provide top, side and bottom views of an example of a cover 700 for an ostomy bag, which can be used with the coupling arrangement of the present invention. The collection bag cover 700 may interchangeably be referred to as the collection bag case. The cover 700 is shaped as a shell, having an open underside (the side configured to sit against the body). The edge of the cover 700 is preferably formed at a shallow angle such that it may achieve a smooth transition to the body. A mount 720 for attachment to the coupling system (specifically, the spring clip 200). The mount 720 (best visible in FIGS. 9b and 9c) comprises a piece of material which extends from the top of the cover 700, proximate the outermost edge of the underside of the cover 700. The mount 720 comprises a central curved section, which corresponds with the curve of the spring clip 300 (in its extended form). The mount 720 further comprises pockets 722 which are configured to accommodate the handles 360 of the spring clip 300. The exterior parts of the pockets 722 extend out of the mount 720, as shown in FIG. 7b. The pockets 722 are less wide than the mount 720, such that the handles 360, when inserted into the pockets 722, are surrounded by the pockets 722 thereby to securely fit the cover 700 to the spring clip 300. FIGS. 9d and 9e show detailed cross-sections of a pocket 722. As will be appreciated, in use the cover 700 is suspended from (or hangs off) the handles 360 of the spring clip 300. This allows the cover 700 to fit securely, while being easily removable for adjustment of the coupling (e.g. when the bag 600 needs emptying).

The spring clip 300 and latch retainer 200 will usually be assembled as a first step in the use of the coupling system. Secure connection of the spring clip 300 and latch retainer 200 is achieved by the engagement of formations such as the protrusion 340, clips 220, 222 and flange 260. FIG. 10 shows an example of the assembly procedure of the spring clip 300 and latch retainer 200. The protrusion 340 may be placed in connection with the rear clips 220 of the latch retainer 200. This connection point then acts as a pivot about which the spring clip is rotated into position, with the protrusion 240 fitting between the rear clips 220 thereby to prevent rotation of the spring clip 300 relative to the latch retainer 200. This is aided by the angle of the edge of the flange 260. Finally, the spring clip 300 can be gently bent under the front clips 222. These steps may be performed prior to attachment to the baseplate 100. As will be appreciated, the various gaps between the clips 220, 222 and the cut-outs in the flange 260 allow this connection to be achieved.

As will further be appreciated, the outer surface 344 of the spring clip 300 presses against the lips 224 of the clips 220, 222 under the urging of the spring clip 300. The clips 220, 222 act to hold the spring clip 300 at the correct position such that the teeth 320 of the spring clip 300 engage with the teeth 142 of the base plate 100. As will be appreciated, the width of the flange portions of the clips 220, 222 is configured to allow compression of the spring clip 300.

The engaged spring clip 300 and latch retainer 200 may then be connected to the baseplate 100 to form the coupling. Pressure is applied to the handles 360, for example in a pinching motion, which causes the spring clip 300 to be compressed. The spacer springs 380 are also compressed such that the effective radius of the spring clip 300 is reduced, while the radius of the central circular portion 280 of the latch retainer 200 remains constant. Under compression, the spring clip 300 and latch retainer 200 may be placed in the baseplate 100 in the channel between the inner circular component 150 and the ratchet mechanism. This is possible because the width of the spring clip 300 is reduced such that the spring clip 300 can pass by the upper lip 132 of the base plate 100 (the width of the flange 260 of the latch retainer 200 is configured such that this can pass by the upper lip 132 of the base plate 100).

Upon removal of pressure to the handles 360, the effective radius of the spring clip 300 returns to its original size due to the outward radial bias. The spring clip teeth 320 and baseplate teeth 142 engage to form a rachet mechanism. In this manner, the spring clip 300 and baseplate 100 become locked together. The spacer springs also become relaxed such that they retain contact with the latch retainer 200. The latch retainer 200 and baseplate 100 form a securely connected continuous conduit, with their circular portions engaging to form a single, preferably fluid-tight, circular tube. The ratchet mechanism may be disengaged by applying pressure on the handles to reduce the effective radius of the spring clip 300. In this manner, the spring clip 300 and latch retainer 200 may be removed from the baseplate 100 or rotated in the channel between the inner circular component 150 and the ratchet mechanism (thereby to allow engagement at a different position, and so re-positioning of the ostomy bag 600).

The spring clip 300, latch retainer 200 and baseplate 100 may be formed of a rigid medical-grade plastic of generally low density. An example of a suitable material is polypropylene. The components are, for example, formed by injection moulding. Additionally, the component parts may further comprise rubber elements, such as for example O-rings, to ensure a water-tight and odour-tight connection. These may, for example, be formed of over moulded thermoplastic rubber (TPR).

The skin adhesive 440 may be selected such that a sufficiently strong bond is formed to ensure that the system is secure when worn, but not so strong that the wearer suffers significant discomfort or skin lacerations when removed. The adhesive is preferably medical-grade, biocompatible, and hypoallergenic. Preferably, the adhesive is a hydrocolloid dressing, which is advantageously breathable but waterproof and allows for normal washing and bathing.

The adhesives 460, 200 used to connect the baseplate 100 to the wafer 400 and the latch retainer 200 to the ostomy bag 600 is selected so as to be strong, water-tight and odour-tight. Alternatively, these adhesives may be replaced by an alternative bonding method such as heat welding.

The cover 700 is preferably formed of silicone, specifically a bio-grade silicone. Even more preferably, the cover 700 has a soft-touch finish. It may advantageously be provided in range of colours, for example most preferably in colours which match skin tones. Optionally, a design (such as a graphic print) or 3D formation is provided on the cover, for decoration.

The present invention advantageously may allow the user to couple and release the system without the need to apply pressure in a direction towards the body. The removal and reapplication of current designs of ostomy systems utilize push-fit couplings. As such, they require pressure to be applied to the body (in particular, directly onto the peristomal skin) in order to secure the coupling. The repeated removal and reapplication can cause discomfort to the user. The coupling system of the present invention may advantageously avoid the need to apply pressure to the body, by instead ensuring that the coupling is releasable from the side. In use, pressure is applied to the handles 360 in a pinching motion to compress the spring clip and release the coupling, and therefore pressure is applied in a plane predominantly parallel to the surface of the body.

As the coupling mechanism of the present invention is intended to be used in ostomy bag systems, the coupling may form a seal which is both water-tight and odour-tight. Additionally, the system is arranged to be compact and light, so that it may be both comfortable and discrete. Accordingly, the system is configured to ensure a snug fit of each connection. This is aided by the use of engagement formations on the components, where examples of such formations include the clips 220, 222, the flange 260, the recesses 180 and the protrusion 340.

FIG. 11a illustrates an alternative example of the present invention. In this example, the latch retainer and spring clip are replaced by a single spring-loaded circular component 1200. The spring-loaded circular component 1200 is outwardly radially biased and engages with the base plate 1100. The coupling may be released by applying force to the handles 1220, and the spring-loaded circular component 1200 then rotated within the base plate 1100 or removed. The spring-loaded circular component 1200 may be snugly placed into position, the handles 1220 being configured to accommodate the shape of the base plate 1100. FIGS. 11 b and c illustrate the system in the locked position, and Figures d and e illustrate the system in the released position. The system preferably further comprises an O-ring 1240 to ensure the seal is water-tight and odour-tight. This may advantageously be formed of rubber such as over-moulded TPR.

FIG. 12a illustrates a further example of the present invention, in which the coupling may be formed of two components, as the latch retainer and spring clip are replaced by a bag clip 2200. The baseplate 2100 and bag clip 2200 may be locked together via the engagement of the protrusions 2220 on the bag clip 2200 and the lock slots 2120 on the baseplate 2100. The engagement may be formed by squeezing the bag clip 2200 at the points of the protrusions 2220. The engagement may be released by squeezing the handles 2240, which releases the protrusions 2200. FIGS. 12b and c illustrate this example in unlocked and locked positions respectively. The system and all alternative examples may advantageously further comprise features to ensure a water-tight and odour-tight seal, as has been previously described.

FIG. 13a illustrates a further example of the present invention, in which coupling may be formed via a screw fit mechanism. This coupling formation comprises a bag clip 3200, which is attached to the collection bag, a baseplate 3100 to be attached to the human body, and a screw clip 3300. The bag clip 3200 may be placed within the baseplate 3100, and then the screw clip 3300 rotated to secure the coupling. Similarly, the clip 3300 may be rotated in the opposite direction to release the coupling. FIG. 13b illustrates the manner in which this example may be unlocked.

FIG. 14a provides a further example of the present invention, in which the coupling system comprises a bag clip 4200 which may be clamped onto the baseplate 4100. The bag clip comprises two clamping edges 4260. The coupling may be secured by placing the bag clip 4200 within the baseplate 4100, and then squeezing the two clamping edges 4260 of the bag clip 4200 such that they clamp around the outer edge of the baseplate 4100. The corresponding tongue 4220 and grooves 4120 are configured to ensure a secure seal, which is preferably water-tight and odour-tight. The bag clip 4200 may further comprise a rubber insert 4240, which provides flexibility to aid in the pivot of the clamping mechanism. FIGS. 14b and c illustrate this example in unlocked and locked positions respectively. The bag clip 4200 is further configured so as to receive the attachment mechanism 4722 of the cover 4700. The attachment mechanism 4722 is comprised of clips within the circular portion of the slit 4720, which may sit between the two clamping edges 4260 to secure the cover 4700 over the coupling.

It will appreciated that various other mechanisms could be used which provide a coupling system which is operable from the side of the coupling. More specifically, various other mechanisms could be used to provide such a system that includes handles which are pinched to release the coupling—examples of such alternative mechanisms are shown in FIG. 15.

FIG. 16 provides a further example of a coupling system, comprising an adhesive wafer 5400, a first member (also referred to as a baseplate) 5100, a ring component (also referred to as a rotating ring) 5300, a further component (also referred to as a bag clip) 5200, and a collection bag 5600 including a deodorising vent 5650. At least one second member (also referred to as an arm) is provided on the further component. As described previously, the wafer 5400 is a thin component formed of fabric or plastic, which is attached to the user's skin via adhesive on one side and the baseplate 4100 on the other side.

The wafer 5400 is attached to the baseplate 5100 via thermal welding or via adhesive as previously discussed. As previously described, the wafer 5400 comprises a skin adhesive for attachment to the body, for example a hydrocolloid dressing that is breathable but waterproof and allows for normal washing and bathing. The wafer 5400 comprises an opening 5480 to receive the stoma, the size and shape of which may be determined and adapted according to the needs of the user. The wafer 5400 further comprises a tab 5420 by which the user can grip the wafer 5400 to remove it.

The baseplate 5100 is a circular component, comprising an inner ring 5150 formed as an inner circular (or tubular) component; a base 5160, and an outer ring 5130, which is located outwardly of inner ring 5150. The base 5160 is a flat, generally ring-shape part which extends from the bottom of the inner ring 5150 to the outer ring 5130, thereby to connect them. The base 5160 is in adhesive contact with the wafer 5400. The inner ring 5150 extends further away from the base 5160 than the outer ring and comprises a wiper seal (that is, a flexible sealing element which is configured to seal against the outer part of a radial element), which is configured to form a sealed fluid conduit with the bag clip 5200 from the opening 5480 to the ostomy bag 5600. The outer ring 5130 comprises a lip extending radially outwards, with a chamfered edge 5135. The baseplate 5100 can be considered to be a first member comprising a circular component, which is configured to be attachable to the bag clip 5200 and thereby the bag 5600.

The rotating ring 5300 has a circular, ring-shaped body 5310 from which extends inwardly discontinuous lower flange tabs 5320 configured to engage with the lip of the outer ring 5130 of the baseplate 5100. On the opposite face of the ring-shaped body 5310 extends discontinuous upper flange tabs 5330, which are L-shaped, such that the tabs have two sides which respectively extend axially and radially inwards with respect to the ring-shaped body 5310. The lower flange tabs 5320 and the upper flange tabs 5330 alternate such that they do not coincide at any point on the circumference of the ring-shaped body 5310. This is to facilitate coupling to the baseplate 5100 and bag clip 5200. The lower flange tabs 5320 can deflect to couple to the baseplate 5100. The upper flange tabs 5330 interact with the bag clip 5200 to lock the coupling. In the illustrated embodiment, there are provided three lower flange tabs 5320 and three upper flange tabs 5330 equally spaced. As such, the lower flange tabs are spaced at 120 degrees from one another, and the upper flange tabs are separated at 120 degrees from one another. Each upper flange tab 5330 comprises a handle 5332, which protrudes a small extent radially outwards from one end such that the handle may be manipulated by a user's finger(s). In the vicinity of each of the upper flange tabs 5330, there is a slot 5312 extending through the body 5310 and the upper flange tab 5330.

The bag clip 5200 is formed of a ring plate 5220, which as the shape of a flat ring and is connected to the collection bag 5600 via heat welding (or alternative method, as has been previously discussed). From the ring plate 5220 extends an inner wall 5222 from the inner edge and an outer wall 5224 from the outer edge. The inner wall 5222 extends further from the ring plate 5220 than the outer wall 5224, and is configured to engage with the wiper seal of the inner ring 5150 of the base plate (such that the wiper seal fills and seals and gap between the inner wall 5222 and the inner ring 5150 of the baseplate—see FIG. 22c in this regard), forming a sealed fluid conduit. Extending radially outwards from the outer wall 5224 are arms 5240 and tabs 5260. The arms 5240 (second members) are biased radially relative to the circular portion of the baseplate 5100 (the first member), thereby to couple the baseplate 5100 to the bag clip 5200. The arms 5240 and tabs 5260 are located next to one another and are configured to coincide with the upper flange tabs 5330 of the rotating ring 5300. As such, in the illustrated embodiment they are provided in three equally spaced sets. The arms 5240 are connected to the outer wall 5224 at one end and extend tangentially to the circular components (for example, the circular portion of the baseplate 5100 and the outer wall 5224 of the bag clip 5200). They are biased radially outwardly with respect to the circular components, and are able to deflect radially inwards around the point of connection. By doing so, they may be deflected and then return resiliently to their original biased position, for example so as to engage with a different component (specifically, the slot 5132 of the rotating ring 5300). At the tip of each of the arms 5240 there is a protrusion 5242 extending radially outwards, which is configured to fit within one of the slots 5312 of the rotating ring 5300.

This arrangement also comprises an ostomy collection bag 5600 being configured to collect bodily wastes as has been previously described, which comprises an opening positioned within the central ring of the bag clip 5200. The bag 5600 further comprises a deodorising filter 5650, which is welded to the collection bag 5600, and comprises an activated charcoal deodoriser and a high airflow membrane.

FIGS. 17a to c illustrate the assembly and interaction of the wafer 5400, the baseplate 5100 and the rotating ring 5300. The wafer 5400 is bonded to the base 5160 of the baseplate 5100 by thermal welding. The first step in assembly of the ostomy coupling system is typically the connection of the rotating ring 5300 to the baseplate 5100. The rotating ring 5300 is snapped over the outer ring 5130 of the baseplate 5100, deforming slightly as is passes over the chamfered edge 5135 of the outer ring 5130, before snapping into position in the groove formed between the chamfered edge 5135 and wafer 5400. The body 5130 of the rotating ring 5100 then sits around the baseplate 5100, and the lower flange tabs 5320 sit below the outer ring 5130. The outer ring 5130 may be continuous, as is illustrated, or may be discontinuous and the baseplate is formed of flexible moulded thermoplastic polyurethane (TPU), which can assist in facilitating the snapping together of the two elements. It can also aid in retaining the rotating ring 5300 in position when connected. The rotating ring 5300 is formed of rigid polypropylene (PP). Once the baseplate 5100 and rotating ring 5300 are snapped together, their connection is secure in that it is very difficult to uncouple them (this may be referred to as a ‘non-return snap fit’); however, the rotating ring 5300 can easily be rotated both clockwise and anti-clockwise with respect to the baseplate 5100. The user can rotate the rotating ring 5300 by gripping the handles 5332. As such, the rotating ring 5300 is attached to the baseplate 5100 via a non-return interaction in that it cannot be moved axially relative to the baseplate 5100 (without a user's specific intervention). However, the rotating ring 5300 is able to freely rotate around the axis of the baseplate 5100.

FIGS. 18a and b illustrate the connection of the bag clip 5200 and the bag 5600, which is formed by thermal welding. The bag 5600 is formed of soft polyurethane (PU) sheets which are welded together, and the bag clip is formed of moulded rigid polypropylene (PP). The bag 5600 further comprises a deodoriser 5650, which includes an activated charcoal carbon deodoriser and a high airflow membrane. Optionally, a further membrane (in addition to that of the deodoriser) may be provided.

FIG. 19 then illustrates the connection of the wafer 5400, baseplate 5100 and rotating ring 5300 as one element worn by the ostomate (the patient/user), where the bag 5600 and bag clip 5200 are a second element. During assembly of the two elements, the user places the arms 5240 and tabs 5260 of the bag clip 5200 within the spaces between the upper flange tabs 5330 of the rotating ring 5300, as illustrated in FIG. 20a. The user then rotates the rotating ring 5300 using the handles 5332 such that the arms 5240 and tabs 5260 are accommodated below the upper flange tabs 5330. The arms 5240 extend from the outer wall 5224 and have a long component which extends approximately parallel to the outer wall 5224 is a direction opposite to the direction of the locking rotation. As such, the flange tabs 5330 encounter the tip of the arms 5240 where are located the protrusions 5242. The arms 5240 are thus deflected radially inwards beneath the upper flange tabs 5330 during this rotation. As the arms 5240 are biased radially outwards, once the slots 5132 on the rotating ring coincide with the position of the protrusions 5242, the arms 5240 spring back outwards and the protrusions 5242 snap into the slots 5132. The connection of the two elements is thus secured via this ‘snap lock’. This is illustrated in FIGS. 21a and b, which show the interaction of the rotating ring 5300 and baseplate 5100 from the side and back. The ‘snap lock’ of the protrusions 5242 and the slots 5312 produces an audible ‘click’ by which the user can tell that connection has been secured. The tabs 5260 can provide further points of contact, which increases the surface area of contact and therefore increases the security of the connection.

The direction of rotation is such that the tab 5260 is encountered first by the upper flange tab 5330 (in the present illustrated example, clockwise). The upper flange tabs 5330 comprise a further tab referred to as a stop edge 5332a, which may be an extension of the handles 5332, as illustrated in FIG. 21b. The tabs 5260 sit adjacent to the stop edges 5332a to prevent further rotation of the rotating ring 5330. This avoids over-rotation, which could result in accidental unlocking of the ‘snap lock’. To disengage the ‘snap lock’, the rotating ring 5300 can be rotated in the opposite direction (in the present example, anti-clockwise) to disengage the bag clip 5200 and remove the bag 5600, for example to be changed for a new one.

FIG. 22a shows the ostomy system from the side when connected to form a continuous and sealed fluid conduit to the ostomy bag 5600. FIG. 22b shows a cross-section across the line A-A as indicated in FIG. 22a, and FIG. 22c shows a magnified view of the region labelled B on FIG. 22b, illustrating the interaction of the baseplate 5100, rotating ring 5300 and bag clip 5200. The baseplate 5100 comprises a flat base 5160 which is connected to the wafer 5400. From the base 5160 extends an inner ring 5150, which fits within a U-shaped groove of the bag clip 5200 formed by the ring plate 5220 (which is connected to the bag 5600), inner wall 5222 and outer wall 5224. The inner ring 5150 of the baseplate 5100 comprises on its inner face a wiper seal 5155, formed of a flexible deformable polymer. When the baseplate 5100 and bag clip 5200 are connected, the inner wall 5222 of the bag clip 5200 compresses the wiper seal 5155 to form a seal which is substantially leak proof.

The baseplate 5100 further comprises an outer ring 5130, and the outer wall 5224 of the bag clip 5200 fits within a U-shaped groove formed by the by inner ring 5150 and outer ring 5130 of the baseplate 5100. The outer ring 5130 has a chambered outer edge 5135, which is configured to guide the rotating ring 5300 over this edge and to snap into place below the outer ring 5130 and around the base 5160. The lower flange tabs 5320 have a chambered inner edges to further facilitate and guide snap connection.

As described for other example ostomy systems of the present invention, it may be desirable to use a cover over the system. FIG. 23 illustrates such a cover 5700 being positioned over the ostomy collection bag 5600 and coupling system. The bag cover 5700 connects with the coupling mechanism to prevent unintentional removal.

FIGS. 24 and 25 illustrates an alternative implementation, in which the baseplate 6100 comprises arms configured to engage with a rotating ring 6300, and the rotating ring is snapped into connection over circular walls of the bag clip 6200. The mechanism of the interaction of the elements otherwise remains unchanged.

Although the rotating ring 5300 and bag clip 5200 are locked relative to one another, they are not locked relative to the baseplate 5100. However, friction in the system, optionally in particular between the bag clip 5100 and the seal 5155, generally prevents rotation of the baseplate 5100 relative to the bag 5600 and bag clip 5200. It will be appreciated, however, that the friction in the system can be adjusted, for example due to the use of different materials or different tolerances, so as to allow rotation of the baseplate 5100 relative to the bag 5600 and bag clip 5200.

Indeed, in some instances, the user may wish to rotate the bag relative to their body, but may not wish to do so in others. Accordingly, optionally a dual-mode functionality may be provided whereby the bag can either freely rotate or lock into place. This could be implemented via a shore-hardness change to the bag clip (which interacts with the wiper seal of the baseplate), optionally such that the bag clip can deform slightly so as to rotate more easily. Specifically, a separate collection bag and bag clip may be provided for each mode (where each bag clip has a different shore hardness) so that a user can change the bag to change between modes. Alternatively, the mode-change may be initiated by a user-controlled switch for alternating between mechanisms. The switch may actuate that an alternative element of a different shore hardness comes into contact with the wiper seal, for example.

In some implementations, in addition to an audible click indicating to a user that the coupling system is locked, the mechanism may also implement a visual indication. This may be indicated by colour, symbols or text. For example, as can best be understood considering FIGS. 20a and b, the upper flange tabs 5330 may comprise a cut-out portion or window, through which a coloured portion on the tab 5260 can be seen only when in the locking orientation.

It will be understood that the present invention has been described above purely by way of example, and modifications of detail can be made within the scope of the invention.

Each feature disclosed in the description, and (where appropriate) the claims and drawings may be provided independently or in any appropriate combination.

Reference numerals appearing in the claims are by way of illustration only and shall have no limiting effect on the scope of the claims.

1. A coupling system for use in medical applications, comprising:
a first member comprising a circular portion; and
a second member being biased radially relative to the circular portion thereby to couple the first member to a further component.

2. A coupling system according to claim 1, further comprising the further component, wherein the further component comprises the second member.

3. A coupling system according to claim 2, wherein the further component further comprises a circular portion; and the second member is an arm extending from the circular portion.

4. A coupling system according to claim 3, wherein the arm is arranged radially outward of the circular portion.

5. A coupling system according to claim 3 or 4, wherein at least part of the arm extends generally tangentially to the circular portion; preferably wherein said extension is in a direction the same as a direction in which the arm is rotated in order to couple the first member to a further component.

6. A coupling system according to any of claims 2 to 5, further comprising a ring component, wherein the second member is configured to bear against the ring component to couple the first member and the further component.

7. A coupling system according to claim 6, wherein the ring component is arranged to rotate relative to the further component.

8. A coupling system according to claim 6 or 7, wherein the ring component comprises a formation for receiving the second member thereby to couple the first member and the further component; preferably wherein the ring component is arranged to be rotated relative to the further component into a position in which the formation receives the second member.

9. A coupling system according to claim 8, wherein the formation receives the second member in a snap fit, preferably wherein the formation and second formation are configured to produce an audible sound when the formation receives the second member; more preferably wherein the system produces a visual indication when the formation receives the second member.

10. A coupling system according to claim 8 or 9, wherein the ring component comprises a flange including the formation, the flange being configured to accommodate and deflect the second member, preferably where the formation is a groove or discontinuity in the flange; more preferably a groove or discontinuity in an outer wall of the flange.

11. A coupling system according to claim 10, wherein the further component further comprises a tab located proximate the second member, wherein the flange comprises a further tab configured to abut the tab thereby to prevent rotation of the ring component relative to the further component beyond a position at which the formation receives the second member; preferably wherein the tab is located adjacent an end of the second member; more preferably wherein the tab is located in a direction relative to the second member that is the same as a direction in which the arm is rotated in order to couple the first member to a further component.

12. A coupling system according to any of claims 8 to 11, wherein the second member comprises a protrusion extending radially from the second member, wherein said protrusion is arranged to be received by the formation; preferably wherein said protrusion is proximate an end of the second member.

13. A coupling system according to any of claims 6 to 12, wherein the ring component is retained by the first member, preferably such that the ring component cannot move in an axial direction relative to the first member.

14. A coupling system according to claim 13, wherein the ring component is retained such that the ring component can rotate with respect to the first member; preferably wherein the ring component is retained in a groove of the first member.

15. A coupling system according to any of claims 6 to 14, wherein the ring component comprises at least one handle; preferably wherein the at least one handle and the further tab are continuous.

16. A coupling system according to any of claims 2 to 15, further comprising a plurality of second members; preferably further comprising a plurality of corresponding formations; more preferably comprising a plurality of tabs.

17. A coupling system according to claim 16, further comprising three second members being arranged symmetrically about the circular portion.

18. A coupling system according to claim 16 or 17 when dependent on claim 9, wherein the flange is discontinuous, preferably such that the continuities correspond to the locations of the second members in a position in which the formations(s) receive the locking members, more preferably thereby to allow the further component to be removed from the first member.

19. A coupling system according to any of claims 2 to 18, wherein the first member and further component couple such that a fluid conduit is formed.

20. A coupling system according to claim 19, wherein one or both of the first member and further component comprises a deformable element being configured to be compressed upon coupling to form a seal.

21. A coupling system according to any of claims 2 to 20, wherein the first member and further component are prevented from rotating relative to each other when coupled, preferably by friction in the seal.

22. A coupling system according to any of claims 2 to 21, wherein the first member and further component are operable to be coupled in a first mode in which the first member and further component are prevented from rotating relative to each other; and a second mode in which the first member and further component are able to rotate relative to each other; preferably: wherein a variant second member and/or further component is provided for use in the second mode; more preferably wherein said variant second member and/or further component has one or more different material properties to the second member and/or further component, and/or wherein the second member is actuable thereby to allow control of the tightness of the seal.

23. A coupling system according to any of claims 2 to 22, wherein the second member is biased radially outwards relative to the circular portion.

24. A coupling system according to claim 1, wherein the second member is arranged to bear against at least one of the first member and a further component thereby to couple the first member to a further component; preferably wherein the second member is arranged to bear against the first member and a further component thereby to couple the first member to a further component.

25. A coupling system according to claim 24, wherein the second member is biased radially outwards relative to the circular portion.

26. A coupling system according to claim 1, 24 or 25, wherein the second member acts as a spring.

27. A coupling system according to claim 26, wherein the second member is shaped as a circlip.

28. A coupling system according to claim 26 or 27, wherein the second member extends at least partially around the first member.

29. A coupling system according to any of claims 26 to 28, wherein the second member further comprises handles which protrude from the coupling system; preferably wherein the handles are located proximate respective ends of the second member, more preferably such that the handles can be pinched to compress the second member.

30. A coupling system according to claim 29, wherein the handles comprise cut-out portions such that other components of the coupling system extend through the cut-out portions.

31. A coupling system according to any of claims 26 to 30, wherein the second member comprises first and second formations for engaging with different components thereby to couple the first member to a further component, preferably wherein the first and second formations are provided on a side of the second member, more preferably wherein the first and second formations are adjacent.

32. A coupling system according to any of claims 26 to 31, wherein the first and second formations are arranged at different widths on the second member.

33. A coupling system according to claim 31 or 32, wherein at least one of the first and second formations comprise teeth, the teeth forming part of a ratchet mechanism.

34. A coupling system according to any of claims 26 to 33, wherein the first member comprises at least one formation with which the second member engages, the at least one formation being located outwardly of the circular portion.

35. A coupling system according to claim 34, wherein the at least one formation comprises at least one clip, preferably wherein the at least one clip extends outwardly from the circular portion; more preferably wherein the width of the at least one clip is configured to allow compression of the second member.

36. A coupling system according to any of claims 26 to 35, wherein the first and second members comprise further cooperative formations which engage thereby to prevent relative rotation between the first and second members.

37. A coupling system according to any of claims 26 to 36, further comprising a third member comprising a circular portion, wherein the third member is the further component; preferably wherein the circular portion is arranged to form a continuous conduit with the circular portion of the first member.

38. A coupling system according to claim 37, wherein the third member further comprises at least one formation against which the second member bears.

39. A coupling system according to claim 38, wherein the formation comprises an outer wall, preferably wherein the outer wall is located outwardly of the conduit.

40. A coupling system according to claim 39, wherein the outer wall comprises teeth forming part of a ratchet mechanism.

41. A coupling system according to any of claims 38 to 40, wherein the third member further comprises a base, preferably extending between the circular conduit and at least one formation, for connecting to a user's skin, preferably via adhesive.

42. A coupling system according to claim 41, wherein a width of the base is configured such that, when compressed, the first and second member can be rotated within a channel created by the gap between the conduit and the at least one formation.

43. A coupling system according to any of claims 26 to 42, wherein the coupling system is for an ostomy bag, wherein the first member attaches to an ostomy bag.

44. An ostomy bag system, comprising the coupling system according to any preceding claim, and an ostomy bag.

45. An ostomy bag system according to claim 45, further comprising a cover for the bag; preferably wherein the cover is suspended from the coupling system of the bag; more preferably wherein the cover comprises formations for receiving corresponding formations of the coupling system; yet more preferably wherein said corresponding formations are handles of the coupling system.

46. A cover for surrounding an ostomy bag, wherein the cover is configured to be suspended from a coupling system of the bag; more preferably wherein the cover comprises formations for receiving corresponding formations of the coupling system.

47. A spring shaped as a circlip for a coupling component for use in medical applications.
